(19) United States
(12) Patent Application Publication
(10) Pub. No.: US 2006/0058879 A1
(43) Pub. Date: Mar. 16, 2006

(54) Vertrebal body placeholder

(76) Inventor: Peter Metz-Stavenhagen, Bad Wildungen (DE)

Correspondence Address: WELSH & KATZ, LTD, 120 S RIVERSIDE PLAZA, 22ND FLOOR, CHICAGO, IL, 60606, US

(21) Appl. No.: 10/515,042

(22) PCT Filed: May 19, 2003
(86) PCT No.: PCT/DE03/01608

§ 371 Date: Jul. 14, 2005

(30) Foreign Application Priority Data
Aug. 24, 2002 (DE) 202-130134.4

(51) Int. Cl. A61F 2/44

(52) U.S. Cl. 623/17.15

(57) ABSTRACT

The invention relates to a vertebral body placeholder comprising a cylindrical inner body (14), which can be inserted in a telescopic manner into a coaxially arranged, sleeve-shaped outer body (16). The aim of the invention is to create a vertebral body placeholder that does not unwantedly penetrate spongy vertebrae. To this end, a cap (20, 22) is placed on a face of the inner body (14) and/or on a face of the outer body (16).

### CROSS-REFERENCE TO RELATED APPLICATIONS

This application claims Priority from German Application No. DE 202 13 013.4 filed on 24. Aug. 2002

### BACKGROUND OF THE INVENTION

1. Field of the Invention

The present invention relates to a vertebral body spacer having a cylindrical inner body that is telescopically slidable into a coaxially arranged, sleeve-shaped outer body.

2. Description of the Prior Art

Such a vertebral body spacer is known from DE 202 07 853.1 for example. In cancellous bones, it may happen that the upper edge of the vertebral body spacer in accordance with DE 202 07 853.1 is pressed into the bone since the contact surface between the vertebral body spacer and the neighbouring vertebral bone is comparably small, resulting in a high surface pressure.

### BRIEF SUMMARY OF THE INVENTION

In view thereof, it is the object of the present invention to provide a vertebral body spacer that does not incidentally penetrate in bones, even if cancellous.

As a technical solution to this problem, it is proposed in accordance with the invention to develop the vertebral body spacer of the type mentioned herein above in such a manner that a cover is mounted to a front side of the inner body and/or to a front side of the outer body.

A vertebral body spacer configured in accordance with this technical teaching has the advantage that the cover provides a large surface on which to support the vertebral bone so that the surface pressure remains low and the vertebral body spacer is no longer allowed to penetrate the vertebral bone.

In order for the cover to be retained properly on the inner body or on the outer body, a preferred developed implementation is suggested in which a guide element engaging the inner body or the outer body is mounted to a face of the cover that is turned toward the inner body or the outer body.

In a preferred embodiment the guide element is configured such that it is retained in the inner body or in the outer body by press-fit engagement. The advantage thereof is that the cover is retained on the vertebral body spacer so as to be secured from being lost also preparatory to implantation without any need for cost-intensive constructions.

In an alternative embodiment, the guide element is configured to be resilient. This also permits to achieve the advantages mentioned herein above, more specifically if one portion of the guide element is configured to be a springy bar projecting at right angles from the cover and coming to rest against an inner side of the inner body or of the outer body.

In still another preferred developed implementation, the cover has a recess through which regrowing bone is allowed to grow into the interior of the vertebral body spacer where it may grow together therewith.

In a particularly preferred embodiment, at least one spike is mounted on the cover. In this case, it has been found advantageous to mount four equidistant spikes thereon. With said spikes, the cover penetrates the neighbouring vertebral bone and prevents the vertebral body spacer from coming out of place.

To fix the cover to the inner or outer body, another preferred embodiment is proposed in which a screw extending through an opening in the inner body or through an opening in the outer body is provided on the cover.

Further advantages of the vertebral body spacer of the invention will become apparent in the appended drawings and in the following description of embodiments thereof. Likewise, the above mentioned features and those described herein after may be used alone or in any combination with each other within the scope of the present invention. The embodiments discussed herein are merely exemplary in nature and are not intended to limit the scope of the invention in any manner.

### BRIEF DESCRIPTION OF THE SEVERAL VIEWS OF THE DRAWING

In the drawing:

FIG. 1 is an exploded perspective view of a spine with a vertebral body spacer of the invention;

FIG. 2 is an exploded side view of the vertebral body spacer according to FIG. 1;

FIG. 3 is a top view of the vertebral body spacer according to FIG. 1;

FIG. 4 is an exploded perspective view of a spine with a second embodiment of a vertebral body spacer of the invention;

FIG. 5 is an exploded view of a third embodiment of a vertebral body spacer of the invention.

### DETAILED DESCRIPTION OF THE INVENTION

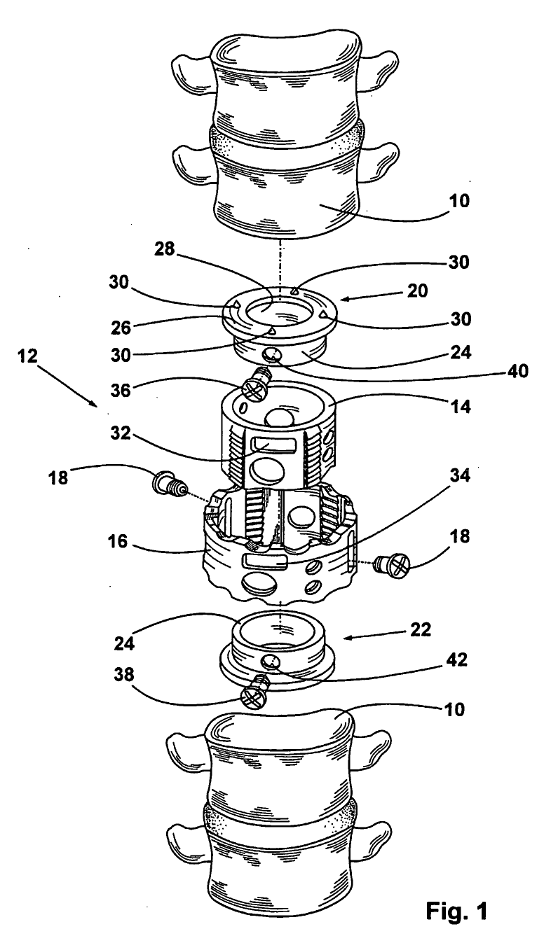

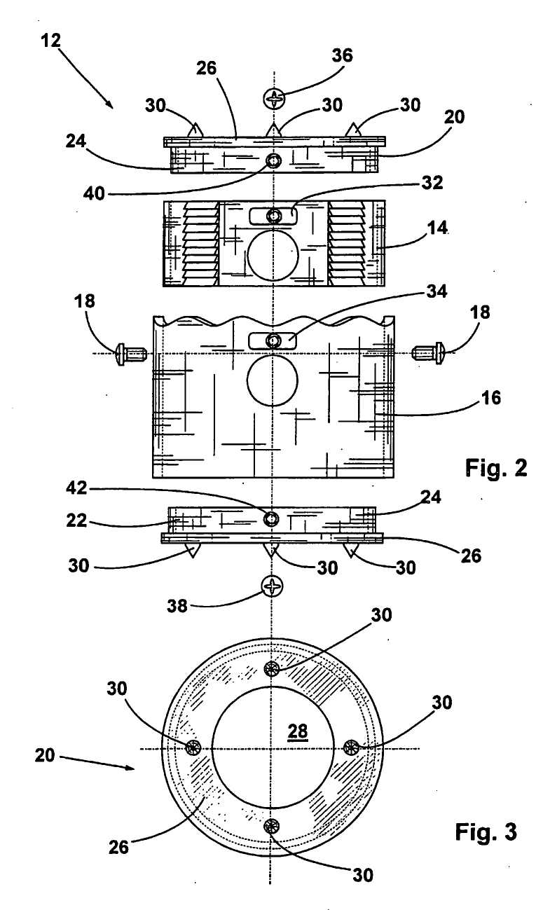

The spine 10 illustrated in the FIGS. 1 through 3 includes various vertebral bodies 10 and a bipartite, cylindrical hollow vertebral body spacer 12 that is mounted between two neighbouring vertebral bodies 10. Said vertebral body spacer 12 is comprised of a smaller, cylindrical inner body 14 and of a larger, sleeve-like outer body 16 which is also cylindrical and positively and telescopically receives the inner body 14. In an effort to achieve an optimal position of the spine, the operating surgeon may fix inner body 14 and outer body 16 in the desired relative position using two fixation screws 18. A long hole 34 arranged in the outer body 16 permits to fix the vertebral body spacer 12 in any position. Above the inner body 14 and beneath the outer body 16 there is provided one cover 20, 22 respectively, said covers being provided with a cylindrical guide means 24 and with an annular cover wall 26 that is disposed perpendicularly to the guide means 24.

In the cover wall 26 there is provided a recess 28 through which the regrowing bone is allowed to grow. On a surface of the cover wall 26 facing the vertebral body 10 there are provided four uniformly spaced spikes 30 for fixing the vertebral body spacer 12 in the confronting vertebral body 10. In the border region of the inner body 14 or of the outer body 16 there are provided long holes 32, 34 through which a fixation screw 34, 36 is insertable. Said fixation screw 34, 36 extends as far as a threaded opening 40, 42 in the cover 20, 22 so as to fix the cover to the inner body 14 or to the outer body 16.

Thanks to the long holes 32, 34, the inner body 14 and the outer body 16 are respectively pivotal about their longitudinal axis in spite of the cover 20, 22 being mounted so as to more specifically operate the detent means that has not been illustrated in closer detail herein. For details concerning this detent means, the reader is referred to DE 202 07 853.1.

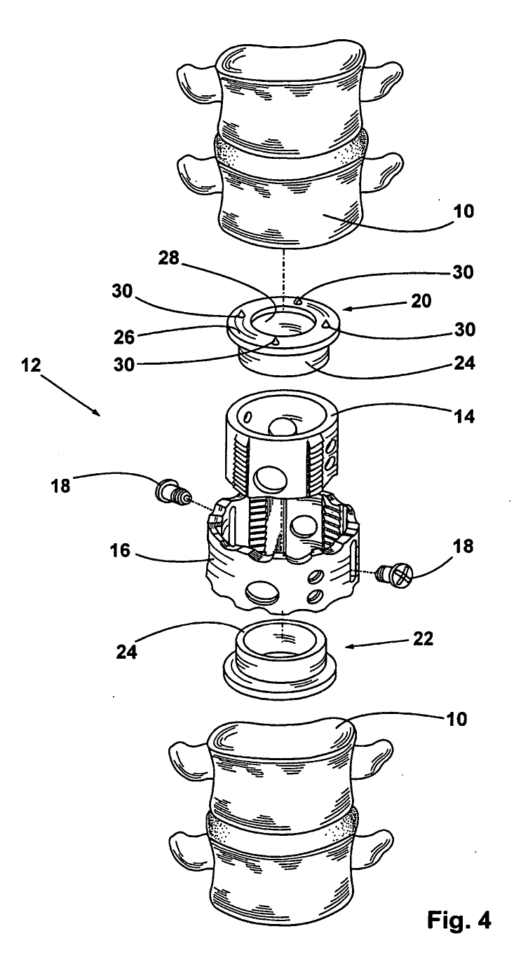

The second embodiment illustrated in FIG. 4 only differs from the first embodiment shown in the FIGS. 1 through 3 by the fact that in this second embodiment the cylindrical guide element 24 is dimensioned such that it is not insertable into the inner 14 or outer body 16 with a clearance but so as to provide a press fit.

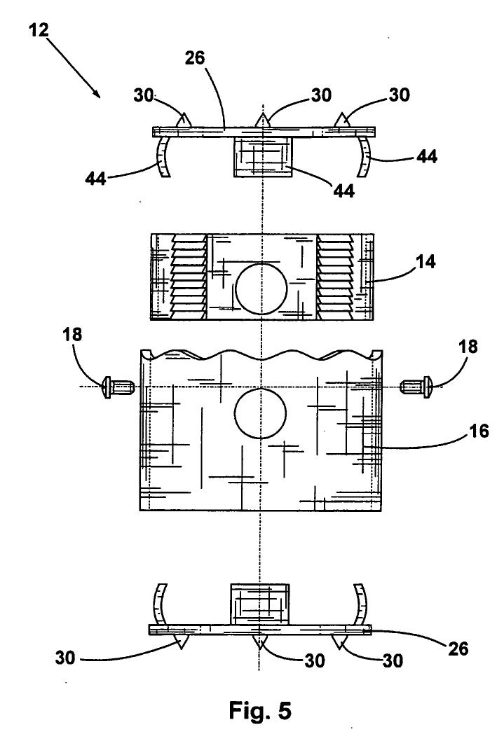

The third embodiment illustrated in FIG. 5 only differs from the first two embodiments illustrated in the FIGS. 1 through 4 by the fact that in this third embodiment the guide element 24 is not configured to be cylindrical but that it comprises four bars 44 perpendicularly projecting from the cover wall 26 and evenly spaced on the circumference thereof. Said bars 44 are formed with an inherently curved shape and are moreover configured to be springy. For inserting the cover into the inner 14 or outer body 16, the inwardly curved portion of the cover is first placed onto the rim of the inner 14 or outer body 16; next, the bars 44 are curved inward by exerting a certain force with the bar material being biased so that the cover fits into the inner or the outer body. From now on, the thus biased bars 44 will exert a resilient force that reliably retains the cover.

### LISTING OF NUMERALS

10 vertebral body

12 vertebral body spacer

14 inner body

16 outer body

18 fixation screw

20 cover

22 cover

24 guide means

26 cover wall

28 recess

30 spike

32 long hole

34 long hole

36 fixation screw

38 fixation screw

40 thread opening

42 thread opening

44 bar

1. (canceled)

2. (canceled)

3. (canceled)

4. (canceled)

5. (canceled)

6. (canceled)

7. (canceled)

8. (canceled)

9. A vertebral body spacer having a cylindrical inner body (14) that is telescopically slidable into a coaxially arranged, sleeve-shaped outer body (16), a cover (20, 22) being mounted to a front side of the inner body (14) and/or to a front side of the outer body (16) and a guide element (24) engaging the inner body (14) or the outer body (16) being mounted to a face of the cover (20, 22) that is turned toward the inner body (14) or the outer body (16), characterized in the guide element (24) in configured to be resilient.

10. The vertebral body spacer according to claim 9, characterized in that the guide element (24) comprises at least one bar (44) that is spring loadable.

11. The vertebral body spacer according to claim 10, characterized in that a recess (28) is provided in the cover (20, 22).

12. The vertebral body spacer according to claim 10, characterized in that at least one, preferably four, spikes (30) are mounted to the cover (20, 22).

13. The vertebral body spacer according to claim 10, characterized in that a fixation screw (36, 38) extending through a long hole (32) in the inner body (14) or through a long hole (34) in the outer body (16) is mountable to the cover (20, 22).
